(19) United States
(12) Patent Application Publication
(10) Pub. No.: US 2023/0023344 A1
(43) Pub. Date: Jan. 26, 2023

(54) BIO-SENSOR SYSTEM FOR MONITORING TISSUE VIBRATION

(71) Applicant: Meta Platforms Technologies, LLC, Menlo Park, CA (US)

(72) Inventors: Yuri Toride, Redmond, WA (US); Doruk Senkal, Kirkland, WA (US); Anton Andreevich Shkel, Aliso Viejo, CA (US); Robert Bruce Darling, Lake Forest Park, WA (US); Andrew Heywood Turner, Sammamish, WA (US); Wynn Dickinson Michael, Portland, WA (US); Sean Diener, Bothell, WA (US)

(21) Appl. No.: 17/866,397

(22) Filed: Jul. 15, 2022

Related U.S. Application Data
(60) Provisional application No. 63/224,336, filed on Jul. 21, 2021.

(51) Int. Cl. A61B 5/00; A61B 5/087; A61B 5/091

(52) U.S. Cl. CPC A61B 5/6803; A61B 5/087; A61B 5/091; A61B 5/4866; A61B 5/7264; A61B 2503/12; A61B 2562/0204

(57) ABSTRACT

A headset comprise a frame and a vibration sensor coupled to the frame. The vibration sensor may be located in a nosepad of the frame, and configured to measure tissue vibrations of a user when the headset of worn by the user. A controller receives a signal corresponding to the measured vibration data from the vibration sensor, and analyzes the received signal to infer a sequence of states of the received signal, such as a sequence of respiratory states. The controller further determines a value of a health metric based upon the inferred sequence of states, e.g., a respiratory rate of the user, and performs an action using the determined value of the health metric.

### CROSS-REFERENCE TO RELATED APPLICATIONS

This application claims the benefit of and priority to U.S. Provisional Application No. 63/224,336, filed on Jul. 21, 2021, the entire contents of which are hereby incorporated by reference for all purposes as if fully set forth herein.

### FIELD OF THE INVENTION

This disclosure relates generally to bio-sensors, and more specifically to a bio-sensor system for monitoring tissue vibrations.

### BACKGROUND

In the field of wearable devices, many applications include collecting data from sensors mounted on the wearable devices that enable assessment of different conditions of a user. Headsets have relatively small form factors and there is limited space for sensors. This can make it difficult to include sensors for monitoring, e.g., health, activity, etc. of a wearer of the headset.

### SUMMARY

In accordance with some embodiments, a headset comprises a frame, a vibration sensor coupled to the frame, and a controller. In some embodiments, the vibration sensor is configured to monitor vibration of a tissue of a user wearing the headset. The controller is configured to receive a signal corresponding to the monitored vibration from the vibration sensor, analyze the received signal to infer a sequence of states of the received signal, determine a value of a health metric based upon the inferred sequence of states, and perform an action using the determined value of the health metric.

In some embodiments, the vibration sensor is located within a nosepad of the frame. For example, in some embodiments, the nosepad comprise an overmold surrounding at least a portion of the vibration sensor. The overmold contains a slot or cavity configured to accommodate the vibration sensor. In some embodiments, the vibration sensor is attached to a spring mounted within the overmold, or to a flexible portion of the overmold.

In some embodiments, the inferred sequence of states corresponds to respiratory states of the user, and the determined health metric value corresponds to a respiratory rate of the user. In some embodiments, the controller uses a first model to classify segments of the received signal into two or more classes corresponding to different stages of respiration, and a second model to infer the sequence of states based upon the classification results generated by the first model. The first model may correspond to a kNN (k-Nearest Neighbors) model, while the second model corresponds to a hidden semi-Markov model (HSMM).

In some embodiments, the controller is further configured to monitor the received signal to detect a predetermined characteristic within the received signal, responsive to detecting the predetermined characteristic, analyze the received signal to identify an event associated with the predetermined characteristic, and to perform an action based upon a type of the identified event. In some embodiments, monitoring of the received signal to detect the predetermined characteristic may occur in parallel with analyzing the received signal to infer a sequence of states of the received signal.

In some embodiments, the identified event corresponds to an eating or drinking action by the user, and the performed action comprises notifying the user of a level of food or fluid consumption of the user.

### BRIEF DESCRIPTION OF THE DRAWINGS

FIG. 1 is a perspective view of a headset implemented as an eyewear device, in accordance with one or more embodiments.

FIG. 2A illustrates an example cross-sectional view of a nose pad of a headset that includes a vibration sensor, in accordance with some embodiments.

FIG. 2B illustrates an example perspective view of a nose pad of a headset that includes a vibration sensor, in accordance with another embodiment.

FIG. 2C illustrates another view of a nosepad of a headset containing a vibration sensor, in accordance with some embodiments.

FIGS. 3A-3C illustrate example designs for increasing a compliance level of the vibration sensor by mounting the vibration sensor to a spring, in accordance with some embodiments.

FIG. 3A illustrates an example cross-sectional view of the vibration sensor within the nosepad attached to a spring within a slot or cavity formed in the overmold, in accordance with some embodiments.

FIG. 3B illustrates an example cross-sectional view of the vibration sensor within the nosepad mounted on a thin, flexible portion of the overmold that functions as a silicone spring, in accordance with some embodiments.

FIG. 3C illustrates an example cross-sectional view of the vibration sensor within the nosepad mounted on a portion of the overmold formed as a cantilever unsupported on one end, in accordance with some embodiments.

FIG. 4 illustrates example graphs of vibrational frequency and amplitude that may be measured by a vibration sensor integrated into a headset worn by the user indicative of different types of user activity, in accordance with some embodiments.

FIG. 5 illustrates an example of analyzing tissue vibration data of a user to identify different respiratory stages, in accordance with some embodiments.

FIG. 6 illustrates a diagram showing how the respiration data of a user may be analyzed using a two-stage segmentation approach, in accordance with some embodiments.

FIG. 7 illustrates a diagram of an HSMM that may be used to perform segmentation based on classification results, in accordance with some embodiments.

FIG. 8 illustrates a block diagram of a system for performing respiratory monitoring and event detection in parallel, in accordance with some embodiments.

FIG. 9 is a flowchart of a method for assessing a user condition with sensors mounted on a wearable device, according to some embodiments.

FIG. 10 is a flowchart of a method for determining a health metric for a user using a vibration sensor mounted a wearable device, in accordance with some embodiments.

FIG. 11 is an example system environment of a headset including an audio system, in accordance with one or more embodiments.

The figures depict embodiments of the present disclosure for purposes of illustration only. One skilled in the art will readily recognize from the following description that alternative embodiments of the structures and methods illustrated herein may be employed without departing from the principles, or benefits touted, of the disclosure described herein.

### DETAILED DESCRIPTION

Embodiments pertaining to a bio-sensor system for monitoring tissue vibration are described herein. As used herein, tissue may include, e.g., skin, cartilage, bone, or some combination thereof. Tissue vibrations may propagate via one or more transduction mechanisms (e.g., cartilage conduction, bone conduction, etc.). The bio-sensor system includes one or more vibration sensors that monitor vibrations of the tissue for health monitoring. The bio-sensor system is at least partially integrated into a headset. For example, the one or more vibration sensors are coupled to and/or integrated within the headset. The bio-sensor system may use a model (e.g., machined learned) and the monitored tissue vibration (and in some cases data from other sensors on the headset) to determine values of one or more health metrics. Health metrics generally describe health and/or habits of the user. The bio-sensor system includes one or more vibration sensors, and a controller.

In some embodiments, the one or more vibration sensors monitor vibration of a tissue of a user wearing the headset. A vibration sensor may be, e.g., an accelerometer, a gyroscope (e.g., 1 axis, 2 axis, and/or three axis), a vibrometer, a contact microphone, some other sensor that can directly and/or indirectly be used to measure tissue vibration, or some combination thereof. The one or more vibration sensors may be coupled to and/or integrated within portions of a headset. For example, in some embodiments, at least one vibration sensor is integrated in a frame of the headset, within a nosepad attached to a frame of the headset, and/or the like.

The controller is configured to determine a value of health metric using the monitored tissue vibrations, wherein the health metric describes health and/or habits of the user. Health metrics may be, e.g. a physiological signal (e.g., heart rate, respiratory rate, etc.), a health event (e.g., heart murmur, abnormal heartbeat/sound), symptom (e.g., early warning for heart attack or seizure), bruxism, estimated caloric intake, emotional state, and/or other metrics that describe health and/or habits of the user, or some combination thereof. The controller may input data from one or more sensors, including the one or more vibration sensors, into a model. The one or more sensors includes the one or more vibration sensors, and may include other sensors on the headset (e.g., IMU, temperature sensor, Photoplethysmography sensor, blood Oxygen sensor, cameras, etc.). The model may be, e.g., a machine-learned model (e.g., trained using a large population of test users) that maps data from the sensors (including the monitored vibrations) to one or more corresponding health metrics. In some embodiments, sensor data from the one or more sensors may be input into a plurality of parallel models. For example, in some embodiments, the sensor data may input into a first model corresponding to a signal classification and segmentation model, and a second model corresponding to a deep learning classification model, where each model is configured to analyze the received sensor data in different ways. For example, the signal classification and segmentation model may be used to monitor the sensor data from the one or more sensors to track a health metric of the user over time (e.g., respiratory rate, heart rate, etc.), while the deep learning classification model is used to detect and classify discrete events (e.g., chewing or drinking by the user).

In some embodiments, the information from the one or more vibration sensors may also be used to determine movement of the user. For example, a 3 axis accelerometer may be used to detect head movement, walking, etc. The controller may be part of the vibration sensor. In some embodiments, the controller is part of the headset. In other embodiments, some or all of the controller is separate from the headset. For example, the controller may be located on a server, smartphone, wearable, or some combination thereof, that is communicatively coupled to the headset.

In some embodiments, a headset includes a frame, a vibration sensor, and a controller. The vibration sensor is coupled to the frame. The vibration sensor is configured to monitor vibration of a tissue of a user wearing the headset. The controller is within the frame. The controller is configured to determine a value of a health metric using the monitored vibration. The controller is also configured to perform an action using the determined value of the health metric.

Embodiments of the invention may include or be implemented in conjunction with an artificial reality system. Artificial reality is a form of reality that has been adjusted in some manner before presentation to a user, which may include, e.g., a virtual reality (VR), an augmented reality (AR), a mixed reality (MR), a hybrid reality, or some combination and/or derivatives thereof. Artificial reality content may include completely generated content or generated content combined with captured (e.g., real-world) content. The artificial reality content may include video, audio, haptic feedback, or some combination thereof, any of which may be presented in a single channel or in multiple channels (such as stereo video that produces a three-dimensional effect to the viewer). Additionally, in some embodiments, artificial reality may also be associated with applications, products, accessories, services, or some combination thereof, that are used to create content in an artificial reality and/or are otherwise used in an artificial reality. The artificial reality system that provides the artificial reality content may be implemented on various platforms, including a wearable device (e.g., headset) connected to a host computer system, a standalone wearable device (e.g., headset), a mobile device or computing system, or any other hardware platform capable of providing artificial reality content to one or more viewers.

### Headset Assembly

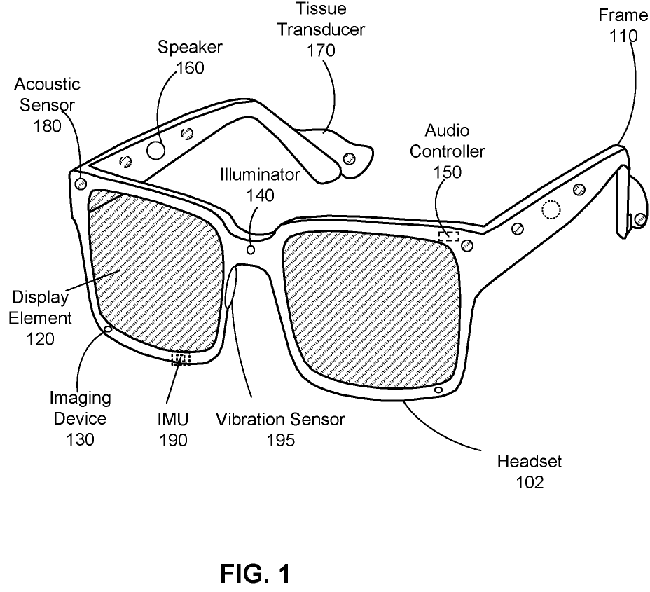

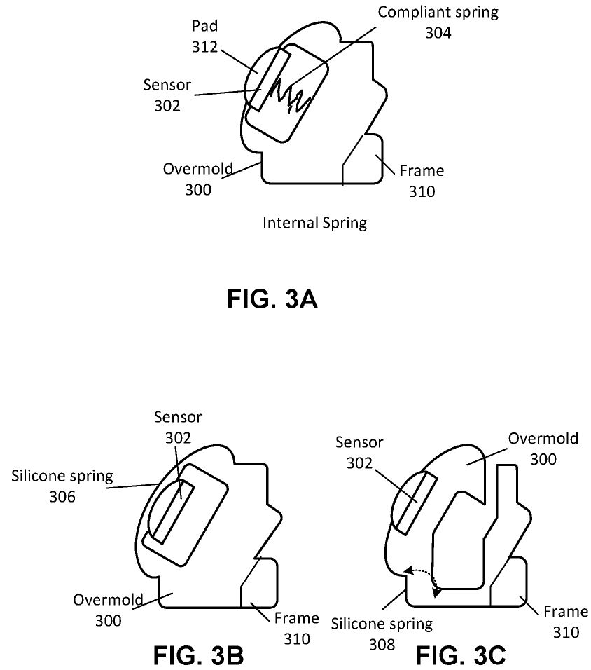

FIG. 1 is a perspective view of a headset 102 implemented as an eyewear device, in accordance with one or more embodiments. In some embodiments, the eyewear device is a near eye display (NED). In general, the headset 100 may be worn on the face of a user such that content (e.g., media content) is presented using a display assembly and/or an audio system. However, the headset 100 may also be used such that media content is presented to a user in a different manner. Examples of media content presented by the headset 100 include one or more images, video, audio, or some combination thereof. The headset 100 includes a frame, and may include, among other components, a display assembly including one or more display elements 120, a depth camera assembly (DCA), an audio system, a position sensor 190, and a bio-sensor system. While FIG. 3 illustrates the components of the headset 100 in example locations on the headset 100, the components may be located elsewhere on the headset 100, on a peripheral device paired with the headset 100, or some combination thereof. Similarly, there may be more or fewer components on the headset 100 than what is shown in FIG. 1.

The frame 110 holds the other components of the headset 100. The frame 110 includes a front part that holds the one or more display elements 120 and end pieces (e.g., temples) to attach to a head of the user. The front part of the frame 110 bridges the top of a nose of the user. The length of the end pieces may be adjustable (e.g., adjustable temple length) to fit different users. The end pieces may also include a portion that curls behind the ear of the user (e.g., temple tip, ear piece).

The one or more display elements 120 provide light to a user wearing the headset 100. As illustrated the headset includes a display element 120 for each eye of a user. In some embodiments, a display element 120 generates image light that is provided to an eyebox of the headset 100. The eyebox is a location in space that an eye of user occupies while wearing the headset 100. For example, a display element 120 may be a waveguide display. A waveguide display includes a light source (e.g., a two-dimensional source, one or more line sources, one or more point sources, etc.) and one or more waveguides. Light from the light source is in-coupled into the one or more waveguides which outputs the light in a manner such that there is pupil replication in an eyebox of the headset 100. In-coupling and/or outcoupling of light from the one or more waveguides may be done using one or more diffraction gratings. In some embodiments, the waveguide display includes a scanning element (e.g., waveguide, mirror, etc.) that scans light from the light source as it is in-coupled into the one or more waveguides. Note that in some embodiments, one or both of the display elements 120 are opaque and do not transmit light from a local area around the headset 100. The local area is the area surrounding the headset 100. For example, the local area may be a room that a user wearing the headset 100 is inside, or the user wearing the headset 100 may be outside and the local area is an outside area. In this context, the headset 100 generates VR content. Alternatively, in some embodiments, one or both of the display elements 120 are at least partially transparent, such that light from the local area may be combined with light from the one or more display elements to produce AR and/or MR content.

In some embodiments, a display element 120 does not generate image light, and instead is a lens that transmits light from the local area to the eyebox. For example, one or both of the display elements 120 may be a lens without correction (non-prescription) or a prescription lens (e.g., single vision, bifocal and trifocal, or progressive) to help correct for defects in a user’s eyesight. In some embodiments, the display element 120 may be polarized and/or tinted to protect the user’s eyes from the sun.

In some embodiments, the display element 120 may include an additional optics block (not shown). The optics block may include one or more optical elements (e.g., lens, Fresnel lens, etc.) that direct light from the display element 120 to the eyebox. The optics block may, e.g., correct for aberrations in some or all of the image content, magnify some or all of the image, or some combination thereof.

The DCA determines depth information for a portion of a local area surrounding the headset 100. The DCA includes one or more imaging devices 130 and a DCA controller (not shown in FIG. 1), and may also include an illuminator 140. In some embodiments, the illuminator 140 illuminates a portion of the local area with light. The light may be, e.g., structured light (e.g., dot pattern, bars, etc.) in the infrared (IR), IR flash for time-of-flight, etc. In some embodiments, the one or more imaging devices 130 capture images of the portion of the local area that include the light from the illuminator 140. As illustrated, FIG. 1 shows a single illuminator 140 and two imaging devices 130. In alternate embodiments, there is no illuminator 140 and at least two imaging devices 130.

The DCA controller computes depth information for the portion of the local area using the captured images and one or more depth determination techniques. The depth determination technique may be, e.g., direct time-of-flight (ToF) depth sensing, indirect ToF depth sensing, structured light, passive stereo analysis, active stereo analysis (uses texture added to the scene by light from the illuminator 140), some other technique to determine depth of a scene, or some combination thereof.

The DCA may include an eye tracking unit that determines eye tracking information. The eye tracking information may comprise information about a position and an orientation of one or both eyes (within their respective eye-boxes). The eye tracking unit may include one or more cameras. The eye tracking unit estimates an angular orientation of one or both eyes based on images captures of one or both eyes by the one or more cameras. In some embodiments, the eye tracking unit may also include one or more illuminators that illuminate one or both eyes with an illumination pattern (e.g., structured light, glints, etc.). The eye tracking unit may use the illumination pattern in the captured images to determine the eye tracking information. The headset 100 may prompt the user to opt in to allow operation of the eye tracking unit. For example, by opting in the headset 100 may detect, store, images of the user’s any or eye tracking information of the user.

The audio system provides audio content. The audio system includes a transducer array, a sensor array, and an audio controller. However, in other embodiments, the audio system may include different and/or additional components. Similarly, in some cases, functionality described with reference to the components of the audio system can be distributed among the components in a different manner than is described here. For example, some or all of the functions of the controller may be performed by a remote server.

The transducer array presents sound to user. The transducer array includes a plurality of transducers. A transducer may be a speaker 160 or a tissue transducer 170 (e.g., a bone conduction transducer or a cartilage conduction transducer). Although the speakers 160 are shown exterior to the frame 110, the speakers 160 may be enclosed in the frame 110. In some embodiments, instead of individual speakers for each ear, the headset 100 includes a speaker array comprising multiple speakers integrated into the frame 110 to improve directionality of presented audio content. The tissue transducer 170 couples to the head of the user and directly vibrates tissue (e.g., bone or cartilage) of the user to generate sound. The number and/or locations of transducers may be different from what is shown in FIG. 3.

The sensor array detects sounds within the local area of the headset 100. The sensor array includes a plurality of acoustic sensors 180. An acoustic sensor 180 captures sounds emitted from one or more sound sources in the local area (e.g., a room). Each acoustic sensor is configured to detect sound and convert the detected sound into an electronic format (analog or digital). The acoustic sensors 180 may be acoustic wave sensors, microphones, sound transducers, or similar sensors that are suitable for detecting sounds.

In some embodiments, one or more acoustic sensors 180 may be placed in an ear canal of each ear (e.g., acting as binaural microphones). In some embodiments, the acoustic sensors 180 may be placed on an exterior surface of the headset 100, placed on an interior surface of the headset 100, separate from the headset 100 (e.g., part of some other device), or some combination thereof. The number and/or locations of acoustic sensors 180 may be different from what is shown in FIG. 3. For example, the number of acoustic detection locations may be increased to increase the amount of audio information collected and the sensitivity and/or accuracy of the information. The acoustic detection locations may be oriented such that the microphone is able to detect sounds in a wide range of directions surrounding the user wearing the headset 100.

The audio controller 150 processes information from the sensor array that describes sounds detected by the sensor array. The audio controller may comprise a processor and a computer-readable storage medium. The audio controller may be configured to generate direction of arrival (DOA) estimates, generate acoustic transfer functions (e.g., array transfer functions and/or head-related transfer functions), track the location of sound sources, form beams in the direction of sound sources, classify sound sources, generate sound filters for the speakers 160, or some combination thereof.

The position sensor 190 generates one or more measurement signals in response to motion of the headset 100. The position sensor 190 may be located on a portion of the frame 110 of the headset 100. The position sensor 190 may include an inertial measurement unit (IMU). Examples of position sensor 190 include: one or more accelerometers, one or more gyroscopes, one or more magnetometers, another suitable type of sensor that detects motion, a type of sensor used for error correction of the IMU, or some combination thereof. The position sensor 190 may be located external to the IMU, internal to the IMU, or some combination thereof.

In some embodiments, the headset 100 may provide for simultaneous localization and mapping (SLAM) for a position of the headset 100 and updating of a model of the local area. For example, the headset 100 may include a passive camera assembly (PCA) that generates color image data. The PCA may include one or more RGB cameras that capture images of some or all of the local area. In some embodiments, some or all of the imaging devices 130 of the DCA may also function as the PCA. The images captured by the PCA and the depth information determined by the DCA may be used to determine parameters of the local area, generate a model of the local area, update a model of the local area, or some combination thereof. Furthermore, the position sensor 190 tracks the position (e.g., location and pose) of the headset 100 within the room.

The bio-sensor system includes one or more vibration sensors (e.g., vibration sensor 195). As shown in FIG. 1, the vibration sensor 195 is in a nose area of the frame 110. In some embodiments, the frame 110 may include a nose pad and the vibration sensor 195 may be integrated into the nose pad. Note that while the vibration sensor 195 is illustrated in the nose area of the frame 110, in other embodiments, one or more vibration sensors may be located in different places on the frame 110 configured to be in contact with a surface of a user’s body when the headset 100 is worn by the user. For example, in some embodiments, the vibration sensor 195 may be implemented as part of the tissue transducer 170, and configured to contact a temple area of the user when the headset 100 is worn by the user. As described above, the biosystem monitors vibrations of the tissue of a user using the one or more vibration sensors, and uses the monitored vibrations to determine one or more health metrics.

In some embodiments, the vibration sensor 195 may be implemented as a voice accelerometer or contact accelerometer. In some embodiments, the vibration sensor 195 may also be referred to as a conduction microphone. Because the vibration sensor 195 is coupled to the tissue of the user (e.g., bone) instead of being air-coupled, the vibration sensor 195 is able to accurately measure tissue vibration of user, within minimal interference from vibrations originating outside the user, such as wind noise. This is in contrast with traditional microphones, which are typically designed to capture airborne vibrations (e.g., sound waves emitted by the user), while seeking to minimize contribution from vibrations within the user, such as teeth grinding, breathing, etc. In some embodiments, the vibration sensor 195 is configured to measure vibrations along a single axis (e.g., an axis orthogonal to a surface on the user’s body over with the vibration sensor 195 rests, such as the side of the user’s nose, hereinafter referred to as z-axis). In other embodiments, the vibration sensor 195 is configured to measure vibrations along more than one axis. In some embodiments, the vibration sensor 195 is configured to directly contact the user’s skin, while in other embodiments, the vibration sensor 195 is coupled to the user’s body via one or more layers of intervening materials, such as a nosepad overmold layer.

### Vibration Sensor Mounting and Hardware

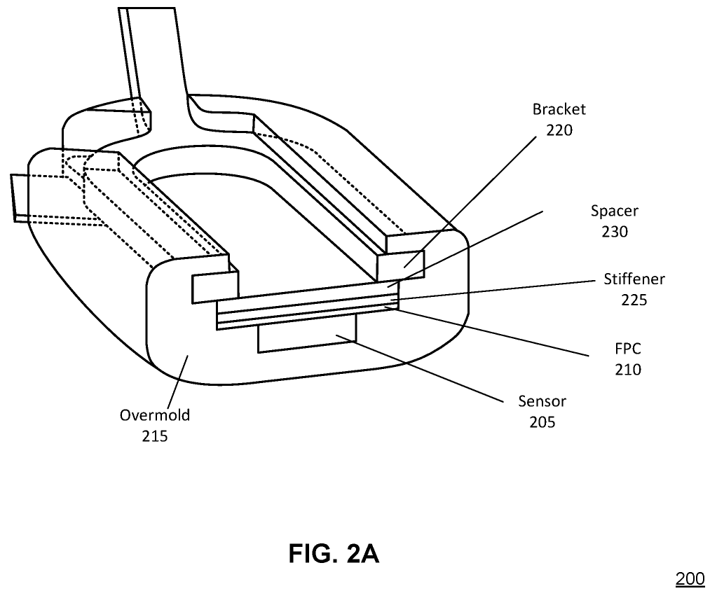

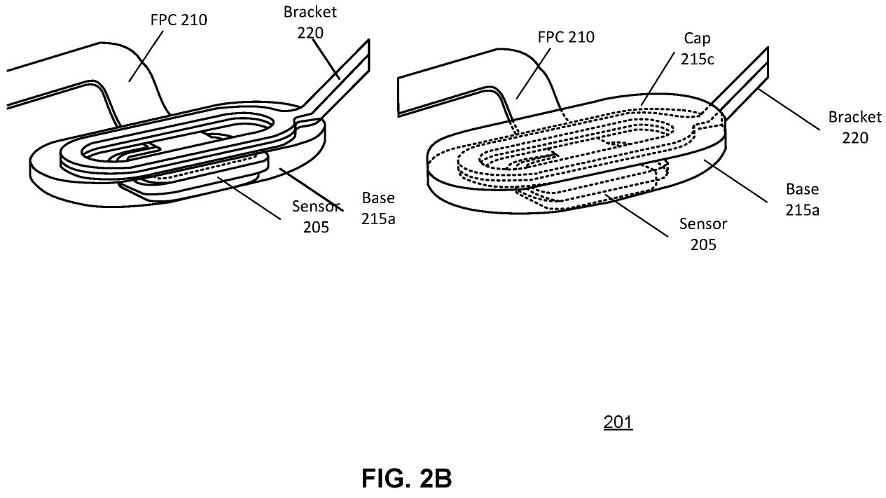

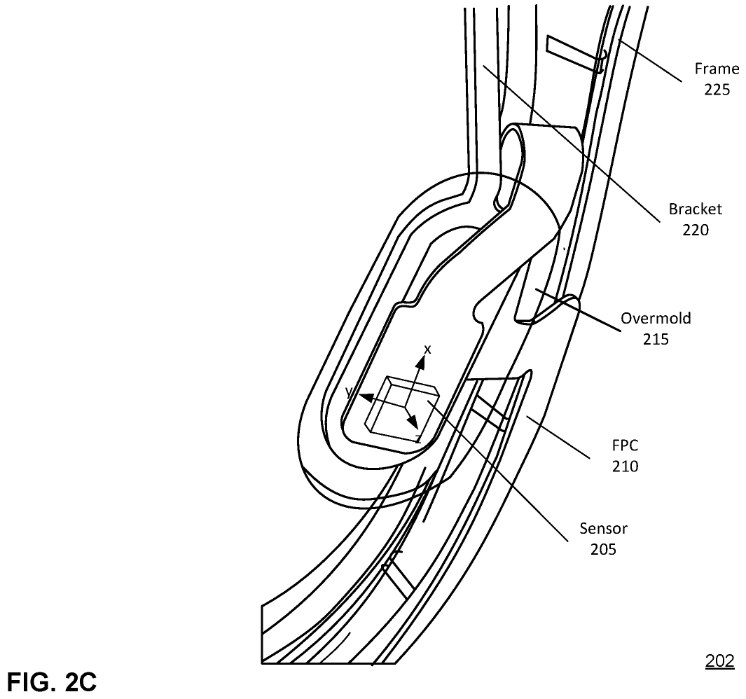

FIGS. 2A-2C illustrate example views of how a vibration sensor may be integrated into a nose pad of a headset, in accordance with some embodiments. In some embodiments, the vibration sensor is integrated into the nosepad of the headset in order to contact a location on the user’s body, such the side of the user’s nose when the headset is worn by the user, directly or via one or more intervening layers, from which vibrational signals indicative of the user’s respiration as well as user activities such as eating, drinking, coughing, etc. can be accurately detected and measured. FIG. 2A illustrates an example cross-sectional view of a nose pad of a headset that includes a vibration sensor, in accordance with some embodiments. In some embodiments, the nose pad assembly 200 includes an overmold 215 that is attached to a frame of the headset via a bracket 220 or other structural components. In some embodiments, the bracket 220 comprises a rigid material such as a metal or hard plastic, while the overmold 215, due to being designed to directly contact the user’s skin when the headset is worn, may comprise a silicone or rubber material, in order to increase comfort of the user.

In some embodiments, such as that illustrated in FIG. 2A, the overmold 215 is formed with one or more slots or cavities in order to accommodate a portion of the bracket 220, as well as additional components such as a vibration sensor. For example, the vibration sensor 205 (which may correspond to the vibration sensor 195 illustrated in FIG. 1) may be mounted on a circuit board, such as flexible printed circuit (FPC) 210, where the vibration sensor 205 and at least a portion of the FPC 210 are accommodated within a slot formed in the overmold 215, such that the overmold 215 surrounds at least a portion of the vibration sensor 205. In some embodiments, the slot may be sized to further accommodate one or more additional components, such as a stiffener 225 for providing additional structural support to the FPC 210 and sensor 205, and/or a spacer 230 between the FPC 210 or stiffener 225 and the bracket 220, that serves to secure the sensor 205 within the slot formed in the overmold 215. As the overmold 215 may be made of a flexible material, such as silicone or rubber, the components accommodated within the slot (e.g., sensor 205, FPC 210, stiffener 225, spacer 230, and/or bracket 220) may be slid into the slot, and held in place by friction. This may allow for different sensors to be swapped in and out of the nosepad (e.g., for repair or replacement), or for a sensor to be swapped between different overmolds (e.g., overmolds of different sizes or materials that may be more comfortable for different users). In other embodiments an adhesive or other material may be used to secure the components within the slot. While FIG. 2A illustrates the nosepad 200 containing a particular combination of components, it is understood that in other embodiments, fewer or additional components may be included. For example, in some embodiments, the sensor 205 and FPC 210 may be accommodated within the slot formed in the overmold 215, without a spacer and/or a stiffener.

FIG. 2B illustrates an example perspective view of a nose pad of a headset that includes a vibration sensor, in accordance with another embodiment. As shown in FIG. 2B, in some embodiments, the overmold may be formed of multiple components, e.g., a base 215a and a cap 215b. In some embodiments, the nosepad is assembled by placing the sensor 205 and at least a portion of the FPC 210 on the base 215a or within a cavity formed in the base 215. In addition, at least a portion of bracket 220 may also be placed on the base 215a or within the cavity formed in the base 215a.

The cap 215b may then be placed over the base 215a, sandwiching the sensor 205, portion of the FPC 210, and portion of the bracket 220 between the base 215a and cap 215b. In some embodiments, the cap 215b is secured to the base 215a, e.g., using an adhesive. In some embodiments, the base 215a and cap 215b may be formed of different materials. For example, the cap 215b, which does not contact the user’s skin when the headset is worn by the user, may be formed from a hard plastic material, while the base 215a, which contacts the user’s skin when the headset is worn, may comprise a silicone or rubber material. In some embodiments, the base 215a and cap 215b are connected at a joint, forming a clamshell design.

FIG. 2C illustrates another view of a nosepad of a headset containing a vibration sensor, in accordance with some embodiments. In some embodiments, the sensor 205 is formed on a side of the FPC 210 facing the user when the headset, and is oriented to lie substantially parallel to a surface of the overmold 215 contacting the user’s skin. In some embodiments, the sensor 205 is configured to measure tissue vibrations of the user in a direction substantially orthogonal to a surface of the user’s skin (e.g., along the z-axis, corresponding to an axis orthogonal to a surface of the sensor 205 configured to be oriented substantially parallel to a surface of the user’s skin with the headset is worn by the user).

As shown in FIG. 2C, the bracket 220 and the FPC 210 extend outside the overmold 215. For example, another end of the bracket 220 (not shown in FIG. 2C) may be secured to the frame 225, fixing a position of the overmold 215 and sensor 205 relative to the frame 225. In addition, the FPC 210 may extend outside the overmold 215 and run along the frame 225, connecting the sensor 205 to a controller of the headset.

While FIGS. 2A-2C illustrate the nosepad connected to the frame of the headset via a bracket, in other embodiments, the nosepad may be an integrated rigid nosepad. For example, in some embodiments, the nosepad overmold may be formed on or attached directly to the frame of the headset. In other embodiments, the nosepad may be correspond to a portion of the frame shaped to rest upon the user’s nose when the headset is worn by the user, without a separate overmold material, where the sensor 205 and its associated components (e.g., FPC 210) are inserted in or otherwise accommodated within a cavity or slot formed in the frame. In addition, it is understood that in other embodiments, the vibration sensor may be located on a different portion of the headset that contacts a surface of the user’s head (directly or via one or more intervening layers) when the headset is worn by the user, such as at a portion of the frame that contacts a temple or side of the user’s head when the headset is worn by the user. By placing the vibration sensor at a location on the headset to measure vibrational data at a location of the user’s head (e.g., nose, temple, etc.), the vibration sensor is able to detect and measure vibrational signals indicative of the user’s respiration as well as user activities such as eating, drinking, coughing, etc.

In some embodiments, the ability of the vibration sensor to capture vibration data of different frequencies is based on a compliance level of the sensor, which may be influenced by how the sensor is integrated into the nosepad. For example, if the vibration sensor is rigidly attached to the nosepad or frame of the headset, the sensor may exhibit low compliance, which may cause the sensor to behave like a high pass filter, and inhibiting an ability of the sensor to detect low frequency vibrations. In some embodiments, to improve the ability of the sensor to capture vibration data corresponding to lower frequencies (e.g., respiratory signals between 300 and 1200 Hz), the vibration sensor may be mounted on a spring, increasing a compliance level of the vibration sensor.

FIGS. 3A-3C illustrate example designs for increasing a compliance level of the vibration sensor by mounting the vibration sensor to a spring, in accordance with some embodiments. For example, in some embodiments, e.g., as shown in FIG. 3A, the sensor 302 accommodated with the overmold 300 may be attached to compliant spring 304 accommodated within a slot or cavity formed in the overmold. In other embodiments, the overmold of the nosepad may be shaped such that a portion of the overmold on which the sensor is mounted is able to flex, forming an elastic silicone spring. For example, as shown in FIG. 3B, the sensor is mounted on a thin, flexible portion of the overmold that functions as silicone spring 306, increasing a compliance level of the sensor. For example, as shown in FIG. 3B, the silicone spring 306 may be able to flex along a direction (e.g., as indicated by a dotted arrow shown in FIG. 3B) orthogonal to a surface of the user’s body contacted by the overmold 300 when the headset is worn by a user, increasing a compliance of the sensor for measuring vibrations from the user’s body. FIG. 3C illustrates another embodiment in which the sensor is mounted on a portion of the overmold formed as a cantilever unsupported on one end, thus functioning as an elastic spring 308. In some embodiments, the sensor 302 may be attached to a pad material 312 different from the material of the surround overmold 300. For example, the pad material 312 may be a material denser than that of the silicone overmold 300, in order to enable a more rigid coupling between the sensor 302 and the user’s skin. In other embodiments, the pad material 312 may be omitted. Although FIGS. 3A-3C illustrate the overmold 300 as directly attached to the frame 310, it is understood that in other embodiments, the overmold 300 may be connected to the frame 310 via a bracket or other component, e.g., as shown in FIGS. 2A-2C.

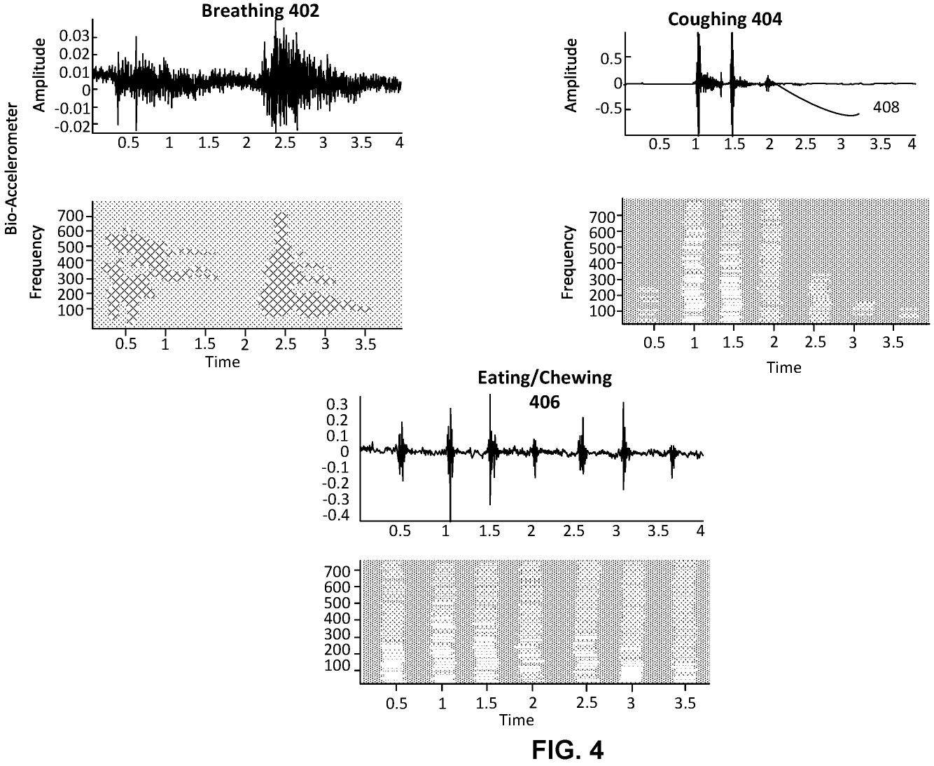

The data captured by the vibration sensor can be used to monitor breathing characteristics of the user, e.g., respiratory rate (RR), breathing depth, etc., as well as detect events exhibited in the behavior of the user, such as coughing/sneezing, eating/chewing, drinking, etc. FIG. 4 illustrates example graphs of vibrational frequency and amplitude that may be measured by a vibration sensor integrated into a headset worn by the user indicative of different types of user activity, in accordance with some embodiments. For example, graph 402 illustrates an amplitude and frequency profile indicating user respiration, while graph 404 illustrates an amplitude and frequency profile of a coughing event, and graph 406 illustrates an amplitude and frequency profile of a user who is eating or chewing. For example, as shown in graph 404, coughing may be characterized by a series of one or more high amplitude impulses 408 having a higher intensity and shorter duration in comparison to normal respiration. In addition, eating/chewing may be distinguished from coughing, as being characterized by more periodic impulses of lower magnitude occurring over a longer period of time.

It is understood that in accordance with some embodiments, analysis of captured vibrational data may be used to identify other types of events based upon amplitude and frequency patterns of the measured vibrations, in addition to those described above in relation to FIGS. 4A-4C. For example, in some embodiments, captured vibrational data may also be used to identify habits of the user such as teeth grinding or nail biting, each of which may be characterized by different vibrational patterns, as well as drinking of fluids. In some embodiments, captured vibrational data may be used to classify detected events. For example, the user’s respiratory patterns may be classified to identify an emotional state of the user (e.g., whether the user is calm or anxious) or an activity state of the user (e.g., if the user is sleeping, exercising, smoking a cigarette, etc.). Similarly, events such as eating/chewing, coughing, etc. may be further classified to identify different types of eating (e.g., whether the user is eating hard or soft food), different types of coughing (e.g., dry cough, wheezing cough, etc.), etc.

In some embodiments, the headset may contain multiple vibration sensors (e.g., a first vibration sensor integrated within a nosepad of the headset, and a second vibration sensor located in a temple region of the headset, and/or different vibration sensors with different properties, such as different compliance levels). In some embodiments, vibration data captured by different vibration sensors is aggregated or cross-referenced during analysis, e.g., when analyzing respiratory characteristics of the user, and/or detecting and classifying events.

### Respiration Monitoring

A user’s respiration characteristics, which may include respiratory (breathing) rate, tidal volume (breathing depth), and/or flow rate, may be a significant predictor of health, wellness, emotional state, and physical activity of user. For example, in many cases, changes in respiratory rate (RR) are sensitive to sudden deteriorations in health, and tidal volume may serve as a useful predictor of cardio-pulmonary complications. If a user’s resting average of either measure rises significantly, this may indicate important changes in health status or possibly problems with air quality in the environment. A user’s RR may also be responsive to stressors (e.g., emotional stress, cognitive load, pain, and fatigue) and can be a good predictor for emotional well-being. Additionally, controlled deep breathing can be very effective for improving mood and reducing stress. A user’s RR may also be sensitive to physical effort and fitness, e.g., where fitter people may show lower respiration rates and faster recovery to resting rates. Tidal volume is correlated to oxygen intake by the body, and is therefore an accurate predictor of physical exertion and recovery.

In some embodiments, a user’s tissue vibration is monitored continuously over a period of time in order to analyze their respiratory patterns (e.g., to determine a resting RR for the user, detect changes in a user’s RR, etc.).

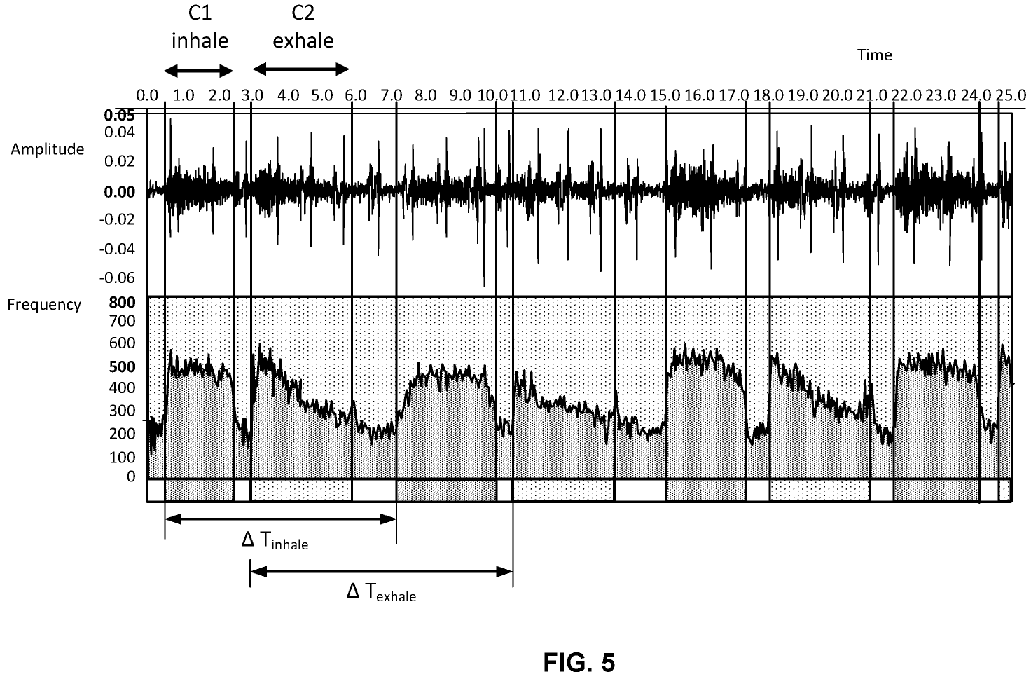

FIG. 5 illustrates an example of analyzing tissue vibration data of a user to identify different respiratory stages, in accordance with some embodiments. A user’s respiratory rate may be analyzed by dividing the captured vibration data into a plurality of classes, each class corresponding to a different phase of breathing. For example, in some embodiments, the vibration data is divided into three classes, corresponding to an inhale phase (shown in FIG. 5 as C1), an exhale phase (shown in FIG. 5 of C2), and a null or silence phase that takes place between inhale and exhale phases. In other embodiments, the vibration data is divided into two classes, corresponding to a breathing phase (encompassing both inhale and exhale) and a null or silence phase.

In some embodiments, segments of vibration data captured by the vibration sensor are labelled with one of a plurality classes (e.g., labels indicating a start and end time of each class), and the respiration rate is calculated based on the times of the identified classes, e.g., ΔTᵢₙₕₐₗₑ = tᵢ[n + 1] - tᵢ[n] corresponding to a time delta between rising edges of consecutive inhale classes (e.g., tᵢ[n + 1] and tᵢ[n]), and ΔTₑₓₕₐₗₑ = tₑ[n + 1] - tₑ[n] corresponding to a time delta between rising edges of consecutive exhale classes.

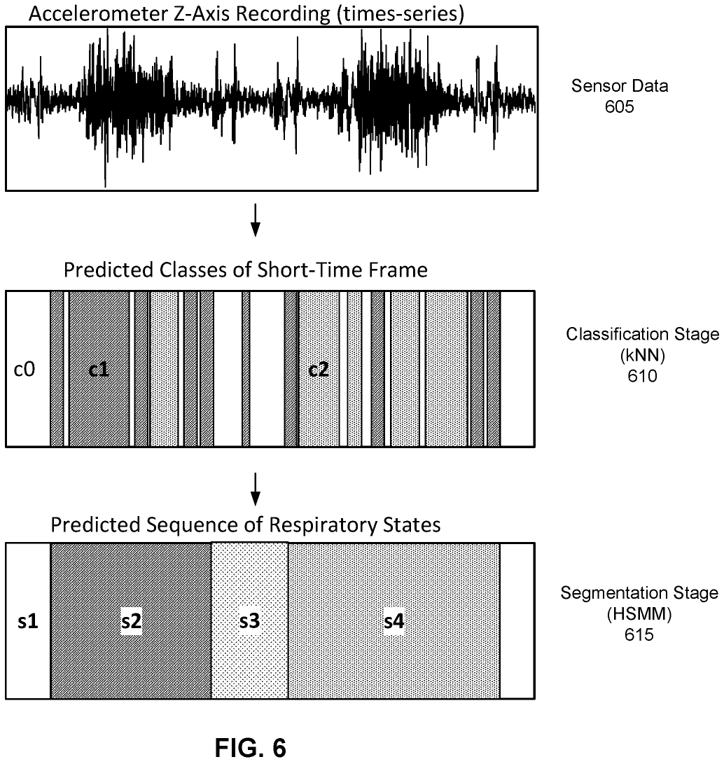

FIG. 6 illustrates a diagram showing how the respiration data of a user may be analyzed using a two-stage segmentation approach, in accordance with some embodiments. During data acquisition, sensor data 605 may be captured as a time-series waveform recording z-axis vibration as detected by a vibration sensor (e.g., a contact accelerometer), and processed through a first classification stage 610 and a second segmentation stage 615. In some embodiments, the sensor data 605 may be pre-processed to remove artifacts (e.g., popping), bandpass filtered to remove noise (e.g., heart noise), and/or combined with additional data to improve a signal-to-noise ratio (SNR) of the signal (e.g., vibration data corresponding to other axes of the accelerometer), e.g., by a pre-processing circuit, prior to being processed by the first classification stage 610 and second segmentation stage 615.

The first classification stage 610 receives the sensor data 605, and classifies segments of the waveform of the received sensor data 605 using a trained model to identify the locations of features (e.g., breathing signatures) present in the vibration waveform indicative of respiration (e.g., inhalation or exhalation). In some embodiments, the first classification stage 610 divides the sensor data waveform into a plurality of narrow frames, computes a set of features for each frame, and use the trained model to classify the feature vector of each frame to determine a class corresponding to the frame, e.g., a breathing class (inhale c1 or exhale c2) or a non-breathing class (e.g., a null class representing all other sounds). In some embodiments, each frame may be of a predetermined length (e.g., 100 ms), and may overlap in time with neighboring frames (e.g., 50% overlap). The trained model is a classifier model trained to recognize differences between frames with and without breathing present, and may be implemented as a kNN model, a Naive Bayes (NB) model, a Support Vector Machine (SVM), and/or the like. In some embodiments, the trained classifier model is trained to classify the frames into three classes (e.g., a no-breathing class c0, an inhale class c1, and an exhale class c2, as illustrated in FIG. 6), while in other embodiments, the trained classifier model is trained to classify the frames into two classes (e.g., a no-breathing class c0, and a breathing class c1 that includes both inhale and exhale).

Because the first classification stage 610 divides the waveform of the sensor data 605 into discrete frames, it may not be sufficient for accurately measuring a respiration rate of the user, as sequence boundaries (e.g., for determining ΔTᵢₙₕₐₗₑ and/or ΔTₑₓₕₐₗₑ as discussed above) not accurately predicted. In addition, the classifier stage has limited understanding of context and temporal sequence outside of individual frames. For example, as shown in FIG. 6, because the classifier model analyzes individual frames (e.g., 100 ms frames), it may classify certain frames as of a non-breathing class c1 between frames of breathing classes (e.g., inhale class c1), leading to potential inaccuracy if determining respiration rate directly from the classified frame data.

Instead, the classification results of the first classification stage 610 are further processed by the second segmentation stage 615, which uses a trained model to infer a most likely sequence of states based on the classification results, as well as a priori knowledge of respiratory cycle characteristics (e.g., durations and transitions). For example, as shown in FIG. 6, the second segmentation stage 615 may infer a sequence of states that includes a null state s1, inhale state s2, pause state s3, and exhale state s4 in sequence, and transition times between each pair of states. In some embodiments, the segmentation model distinguishes between the null state s1 (referring to a period following an exhale until a next inhale cycle) and the pause state s3 (referring to the gap between inhale and exhale), as even though the two states generally exhibit similar frequency and amplitude characteristics, the pause state, being between inhale and exhale, may exhibit a more predictable duration in comparison to null state, which, being between exhale and inhale, may exhibit larger variations in duration. In other embodiments, the null state s1 and pause state s3 may be represented using a single no-breathing state instead of being separate states. In some embodiments, the trained model used by the second segmentation stage 615 is a Hidden Semi-Markov Model (HSMM) trained to predict a sequence of states of maximum likelihood, based on the first stage classification results.

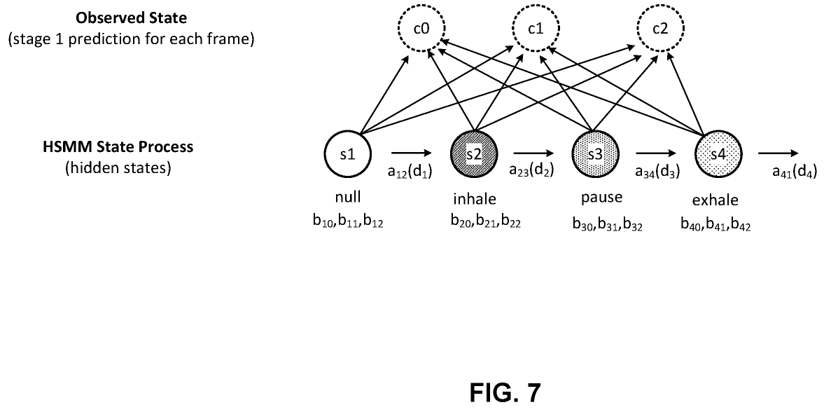

FIG. 7 illustrates a diagram of an HSMM that may be used to perform segmentation based on classification results, in accordance with some embodiments. The HSMM model 700 is composed of four hidden states, which may be described using three parameters: a transmission matrix A, an emission matrix B, and a duration vector d.

The Transmission Matrix A governs the probability of transitioning from any hidden state to another (e.g., from S1 null state to S2 inhale state, from S2 inhale state to S3 pause state, etc.). In some embodiments, the transmission matrix A may be extracted from state sequences in training annotations. In other embodiments, the states are labelled such that each state is sequential and transition probabilities are a function of duration and the probabilities indicated by the emission matrix B. The Emission Matrix B is configured to, in a multinominal emission model, capture a probability of encountering each observation (e.g., class as determined by the first classification stage) at each hidden state, and may be trained based upon an accuracy of a prediction generated by the classifier model during the first classification stage 610. The Duration Vector d characterizes the probability distribution of durations spent in each hidden state. In some embodiments, the duration vector is extracted statistically by fitting training data to a log-normal distribution function.

In some embodiments, the HSMM predicts a state sequence for recorded vibration data by maximizing a probability of the state sequence, based on the parameters above (e.g., by determining a sequence of states that maximizes the transition probability A between each pair of states). For example, in some embodiments, a maximum likelihood estimation is made based upon the information in A, B, and the observed sequence of states output by the classifier model (e.g., as part of the classification stage 610). As shown in FIG. 7, each segment may be determined based upon prior and future observations of the classifier. The transmission matrix A is a function of the duration vector d, so that the state transition probabilities reflect a reasonable rate of state durations. The emission matrix B specifies the probabilities of a state given the observed states output by the classifier, and is used to modify the transmission matrix A to determine the maximize likelihood estimation specifying the timing of state transitions within the observed vibrational data, from which the sequence of states and state durations can be determined. Once a state sequence has been predicted, a respiratory rate of the user may be estimated based upon a period of time between repeated states (e.g., ΔTᵢₙₕₐₗₑ and/or ΔTₑₓₕₐₗₑ).

By measuring the user’s RR, the controller may be able to determine a number of different health metrics pertaining to the user. For example, as discussed above, the controller may monitor the user’s RR over time to determine a resting RR, detect periods of activity by the user (e.g., corresponding to deviations from the user’s resting RR, such as exercise, which may correspond to increased RR, or sleep, which may correspond to decreased RR), identify trends in the user’s RR, and/or the like. In some embodiments, the user’s RR may be used in combination with other respiratory measures such as tidal volume and/or flow rate, to determine one or more respiration-related health metrics for the user. Using this information, the controller may be able to make inferences relating to the user’s current condition and/or general state of health. In addition, in some embodiments, the controller may determine information usable to provide feedback to the user, e.g., notify the user of one or more determined health trends, notify the user of a current activity intensity, instruct the user to perform one or more actions to control their respiration (e.g., as part of a meditation exercise), select appropriate media to be played to the user based on the user’s physical or emotional state (e.g., music to pump the user up during exercise, or to calm the user when the user is anxious).

### Event Detection and Classification

In some embodiments, a user’s recorded vibration data may further be used to detect specific types of events, such as coughing, sneezing, eating, chewing, and/or drinking. In some embodiments, the controller uses a deep learning model, such as a convolutional neural network (CNN), gated recurrent unit (GRU), long short-term memory (LSTM) model, etc., to identify a type of event that occurred, based upon the received vibration data. Unlike breathing/respiration, such events typically do not occur continuously, and instead are more likely to occur sporadically over a monitored time period. As continuously processing received vibration data using a deep learning model to identify events may be computationally demanding and power intensive, in some embodiments, the controller instead monitors the received vibration signal for a trigger event (e.g., similar to a wake word used in speech recognition applications). For example, in some embodiments, a trigger event may correspond to the received vibration signal exhibiting a particular pattern, exceeding a threshold amplitude, exhibiting a frequency within a predetermined range, or some combination thereof (e.g., vibration within a predefined frequency range exceeding a threshold amplitude over a certain period of time). Once a trigger event is detected, recorded vibration data corresponding to a time period associated with the trigger event (e.g., a surrounding time period) is analyzed using the deep learning model to determine a type of event that occurred.

In some embodiments, the deep learning model is trained to, in addition to identifying a type of event reflected in the vibration data, classify the event into one or more sub-types. For example, the user may perform different types of chewing, e.g., based upon a type of food being consumed. As different types of food (e.g., soft food, crunchy food, etc.) may result in different sounds, the deep learning model may be trained to identify a type of food being consumed by the user responsive to determining that the user is chewing. In some embodiments, the event is classified based upon one or more of a frequency content, amplitude, and duration of the identified portion of the vibration data corresponding to the event. In some embodiments, an identified event may be classified (e.g., classification of a chewing/eating event based on different food types and/or ingestion methods) may be performed using a decision tree model, based on the spectral and temporal features of the identified event.

In some embodiments, the deep learning model may further determine an intake amount associated with an identified event (e.g., how much food is being consumed by the user during a detected chewing event, how much fluid is being consumer by the user during a detected drinking event, etc.). For example, the deep learning model may, responsive to detecting a drinking event, determine a number of gulps or sips performed by the user during the detected drinking event, and estimate an amount of fluid being consumed by the user, based upon the determined number of gulps or sips.

In some embodiments, the system may analyze the received event to generate one or more health recommendations for the user. For example, in some embodiments, through the detection of drinking events by the user, the system determines how much time has passed since the user has last drank, and may notify the user responsive to a determination that the user has not drunk any fluids within a threshold period of time (e.g., within the last four hours). In addition, in some embodiments, the system may estimate an amount of water drunk by the user based on analysis of detected drinking events, and provide a recommendation to the user, such as informing the user how close they are to achieving a recommended daily fluid intake amount. In some embodiments, by detecting eating/chewing events by the user, the system may determine an amount of food intake by the user, and/or types of food consumed by the user (e.g., determined based upon a combination of frequency content, amplitude, and duration of the vibration signal, using a decision tree model).

In some embodiments, the system analyzes received events over time to monitor one or more habits of the user. For example, the system may infer eating or drinking habits of the user, based upon a timing at which the system typically detects eating or drinking events by the user, the types of eating or drinking events performed by the user, or some combination thereof. The system may also determine habits of the user associated with other types of events, such as nail biting or teeth grinding, which may be cross-referenced with other data (e.g., respiratory rate data) to determine a physical or emotional condition of the user (e.g., stress, anxiety, etc.). Health Monitoring Using Multi-Sensor Combinations

In some embodiments, the headset contains additional sensors that may be used in combination with the vibration sensor to monitor different aspects of the user’s health. For example, as discussed above, the vibration sensor may be used to detect subtle vibrations at the nose, mouth, and head of the user, which may be indicative of a user health. In some embodiments, the vibration sensor may be used in conjunction with acoustic microphones, where the vibration sensor may trigger an acoustic microphone when an initial event (e.g., a cough event) is detected, or as a double verification that a signal is a veritable health indicator. IMU sensors also may detect sudden movements (e.g., during sneezing or coughing), which combined with a temperature measurement from a thermometer (located on the arm of the smart glass, in close proximity or contact with the back of the user’s head, or the ear lobe), thus detecting a cough and a fever and the onset of an infection, flu or other disease with the user.

In some embodiments, data captured by the vibrational sensor may be cross-referenced with data captured by other types of sensors, in order to determine a health status of the user. For example, the headset may contain an acoustic sensor configured to measure a speech signal of the user, which may exhibit shifts in tone depending on the user’s health status (e.g., the tone of the user’s voice as detected by the acoustic sensor may shift when the user has a stuffy nose). This data may be cross-referenced with events detected through analyzing vibrational data (e.g., the user coughing or sneezing) to infer an overall health status of the user (e.g., that the user has a cold).

In some embodiments, a photoplethysmography (PPG) sensor may be configured to provide an optical measurement of blood vessels and capillaries to provide a heart rate monitoring, pulse oximetry readings, and the like. PPG sensors as disclosed herein may provide waveforms indicative of various cardiovascular-related diseases such as atherosclerosis and arterial stiffness, among others, which may be combined with respiratory information (e.g., respiratory rate, tidal depth, etc.) determined using a vibration sensor to assess an overall cardiovascular health condition of the user.

In some embodiments, the event detection functionality using the vibration sensor may be used to activate other types of sensors or input devices. For example, responsive to detecting certain types of events, such as chewing, the system may activate a camera located on the headset or on another device to capture an image of the food being consumed. Alternatively, the controller may communicate with a user device (e.g., a mobile device of the user) to prompt the user to input information relating to what they are eating. The information identifying the type of food being consumed by the user may be used, in conjunction with the detected chewing event information, be used to perform eating volume classification including a caloric intake estimate for the user.

Some of the health conditions that may be assessed using the combination of sensors illustrated may include asthma monitoring, long term-cough detection, and other chronic respiratory conditions (e.g., associated with Corona Virus -COVID-, pneumonia, seasonal flu, and the like) that may be detected via sensitive motion/acoustic sensors (e.g., environmental health). One or more combinations of sensors may further be used for wellness and fitness applications loaded either on the smart glass or to the associated mobile device, by measurement of breath rate for stress detection. These measurements may be also integrated into meditation, calming, and other psychological/psychiatric support applications.

### Parallel Processing Pipelines

As discussed above, the vibration data received from the vibration sensor may be used to perform continuous health monitoring (e.g., respiration monitoring), as well as detect specific types of events (e.g., coughing, sneezing, chewing, drinking, etc.). In some embodiments, these two functions may be performed in parallel through separate processing pipelines. For example, the vibration data generated by the vibration sensor may be analyzed continuously to monitoring a respiratory rate of the user, while also being monitored for trigger events for triggering a deep learning model for performing event identification and/or classification.

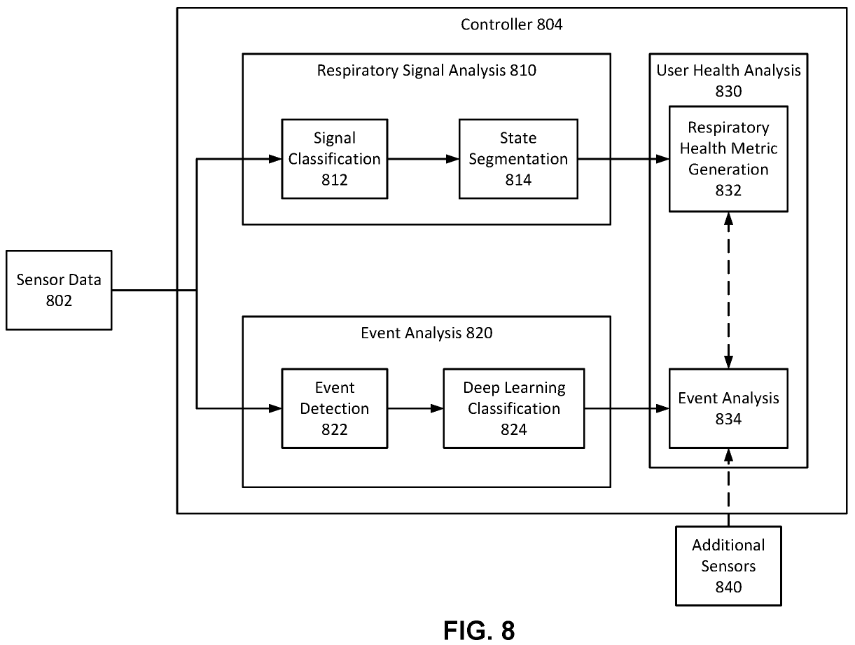

FIG. 8 illustrates a block diagram of a system for performing respiratory monitoring and event detection in parallel, in accordance with some embodiments. As shown in FIG. 8, a controller 804 may receive sensor data 802 (e.g., vibration data) from a vibration sensor, such as that illustrated in FIGS. 1 and 2A-2C. The sensor data 802 is processed through different processing pipelines that perform different types of analysis on the sensor data 802 to generate various metrics relating to a health of the user. For example, as illustrated in FIG. 8, the sensor data 802 is processed through a respiratory signal analysis module 810 and an event analysis module 820 in parallel, to generate different types of information usable by the user health analysis module 830 to analyze the user’s health (e.g., by generating a respiratory health metric for the user, analyzing events associated with the user, etc.).

In some embodiments, the controller 804 comprises one or more processors implemented on a headset, e.g., the headset 100 illustrated in FIG. 1. In some embodiments, the controller may comprise one or more processors located on a device external to the headset, e.g., an external console, mobile device, or server in communication with the headset. While FIG. 8 illustrates a single controller 804, it is understood that in some embodiments, different operations of the controller 804 may be performed by different processors and/or on different devices. For example, in some embodiments, operations of the respiratory signal analysis module 810 may be performed by a processor on a headset, while operations of the user health analysis module 830 are performed by a separate processor on a separate device, e.g., a mobile device.

The respiratory signal analysis module 810 is configured to analyze the sensor data 802 to determine respiratory metrics relating to the user (e.g., the user’s respiratory rate, tidal depth, etc.). Because the user is expected to be continuously breathing during the monitored time period, the respiratory signal analysis module 810 may analyze substantially all of the sensor data 802 to determine the respiratory metrics. In some embodiments, the respiratory signal analysis module 810 processes the sensor data 802 in two stages, using a signal classification model 812 and a state segmentation model 814. The signal classification model 812 divides the sensor data 802 into a plurality of frames, and analyzes the features of each frame to classify the frame as one of a plurality of predetermined states (e.g., a breathing state or a non-breathing state). In some embodiments, the signal classification model 812 corresponds to the classification stage 610 illustrated in FIG. 6, and may be implemented as a kNN model.

The state segmentation model 814 is configured to infer a most likely sequence of states based on the classification results generated by the signal classification model 812, as well as a priori knowledge of respiratory cycle characteristics, and may correspond to the segmentation stage 615 illustrated in FIG. 6, and implemented as a Hidden Semi-Markov Model (HSMM), such as that illustrated in FIG. 7. For example, the state segmentation model 814 may predict transition points between different states of the classified sensor data based upon the classified frames generated by the signal classification model 812, from which a respiratory rate of the user may be determined (e.g., based on an amount of time between repeated states). The respiratory rate data may be used by the user health analysis module 830 to generate one or more respiratory health metrics (e.g., via respiratory health metric generation module 832). For example, in some embodiments, the respiratory rate of the user may be monitored to establish a resting respiratory rate of the user, and/or analyze changes to the user’s respiratory rate (e.g., due to health conditions, stress, and/or physical activity). In some embodiments, the user’s respiratory rate may be used to infer a current physical status of the user (e.g., resting, asleep, exercising, etc., and/or whether the user is experiencing any respiratory-related symptoms, such as respiratory obstruction, inflammation, or pulmonary edema, causing abnormal breathing patterns such as rhonchus, crackle, or shortness of breath, etc.), or an emotional status of the user (e.g., whether the user is currently anxious or suffering a panic attack). In addition, trends in the user’s respiratory patterns may be indicative of an overall change in health status of the user. In some embodiments, the system may detect changes in the user’s respiration that are indications of the offset of certain symptoms (e.g., heart attack/seizure, regurgitation, stenosis, etc.).

In some embodiments, an application may receive respiration information determined for the user, and provide the user with feedback relating to their respiration, e.g., inform the user of an intensity at which they are exercising, providing feedback to the user on controlling their RR as part of a meditation exercise, dynamically selecting media to be played to the user based on a determined anxiety level of the user (e.g., calming music when the user’s anxiety level exceeds a predetermined threshold), informing the user of the possible onset of certain symptoms and recommending preventative action, etc.

In some embodiments, the respiratory signal analysis module 810 is further configured to determine other types of respiratory characteristics of the user (e.g., tidal volume, flow rate) using one or more additional models, such as a regression model (not shown in FIG. 8), to analyze the received sensor data 802 (e.g., a first model that determines a tidal volume of the user’s respiration, and a second model that determines a flow rate of the user’s respiration). In some embodiments, these models are supplemented or cross-referenced with the sequence of states generated by the state segmentation model 814, or receive the generated sequence of states as an input, to improve a quality of the determined respiratory characteristics, such as by correlating a duration of the user’s inhale state with breathing volume. In some embodiments, the health metric generation module 832 generates respiratory health metrics based on a combination of the user’s respiratory rate, tidal volume, and/or flow rate, e.g., to infer a physical or emotional status of the user. For example, a determined health metric indicating an exercise intensity level experienced by the user, or an anxiety level of the user, may be determined based on multiple respiratory characteristics including respiratory rate, tidal volume, and flow rate.

The event analysis module 820 is configured to predict occurrences of specific types of events reflected in the user’s vibration data, and comprises an event detection module 822 and a deep learning classification model 824. The event detection module 822 is configured to monitor the sensor data 802, and to detect the presence of predetermined trigger events indicative of events of interests. In some embodiments, a trigger event may correspond to a predetermined vibrational pattern having amplitude and frequency characteristics within a predetermined range. Because use of deep learning may be computationally demanding and power intensive, the event analysis module 820 may be configured to use the deep learning classification model 824 to analyzed received vibration data only when a predetermined trigger event indicative of an event of interest has been detected by the event detection module 822. Responsive to detecting a trigger event, the deep learning classification model 824 analyzes a portion of the sensor data 805 associated with the trigger event, and predicts a type of event that occurred reflected in the vibration data. In some embodiments, the event detection module 822 may detect different types of trigger events corresponding to different event types, and the deep learning classification model 824 may take into account the type of detected trigger event when predicting the event type. For example, in some embodiments, coughing events may be associated with a first type of trigger event, while chewing events are associated with a second different type of trigger event. In some embodiments, the deep learning classification model 824, in addition to predicting a type of event, may also determine one or more attributes of the event, e.g., the magnitude of a detected cough or sneeze, a type of food associated with a chewing event, and/or the like. In some embodiments, the deep learning classification model 824 may comprise a plurality of different models, each trained to classify a different type of event. For example, in some embodiments, the deep learning classification model 824 may select one or more specific trained classification models, based on one or more characteristics of a trigger event detected by event detection module 822.

The event analysis module 834 of the user health analysis module 830 receives event information from the deep learning classification model 824, such as event type information, event attribute information, etc., and analyzes the received event information to generate one or more health metrics for the user. For example, in some embodiments, the event analysis module 834 may monitor an occurrence frequency of certain event types to determine a health condition of the user, e.g., monitor user sneezing frequency to determine if the user is suffering from allergies, monitor user drinking events to determining if the user is consuming enough fluids, etc. In some embodiments, the event analysis module 834 analyzes the received event information in conjunction with data from one or more additional sensors 840. For example, as discussed above, vibration data may be used in conjunction with data captured using acoustic microphone and/or an IMU to evaluate the magnitude of a cough or sneeze. In some embodiments, the event analysis module 834 may access data from different sensors depending on the type of event that was detected (e.g., a first set of sensors responsive to a detected sneeze event, and a second different set of sensors responsive to a detected chewing event).

In some embodiments, the user health analysis module 830 is configured to perform one or more actions based upon one or more health metrics generated by the respiratory health metric generation module 832 and/or the event analysis module 834, and/or responsive to one or more identified events. For example, the user health analysis module 830 may provide one or more recommendations to the user based on a respiratory health metric of the user, such as providing recommendations for the user to regulating their respiratory rate as part of a mediation exercise, providing exercise-related recommendations following a period where the user’s respiratory rate and tidal depth are indicative of activity associated with physical exercise, alerting the user to abnormal respiratory activity and recommending preventative measures, and/or the like. In addition, the user health analysis module 830 may also perform one or more actions to the user based upon one or more identified events, e.g., generating a report indicating the user’s current daily fluid intake following detection of a drinking event, recommending a treatment option responsive to detection of a sneezing event and/or multiple sneezing events occurring within a threshold period of time, etc. In some embodiments, the user health analysis module 830 may combine or cross-reference health metrics relating the user’s monitored respiration and those relating to detected events to determine one or more overall health metrics for the user and/or detect symptoms of the user. For example, health metrics relating to the user’s respiration (e.g., respiratory rate and/or tidal depth), combined with metrics relating to a frequency and magnitude of coughing/sneezing events by the user, may be used generate a profile indicating an overall cardiovascular health of the user.

### Process Flow

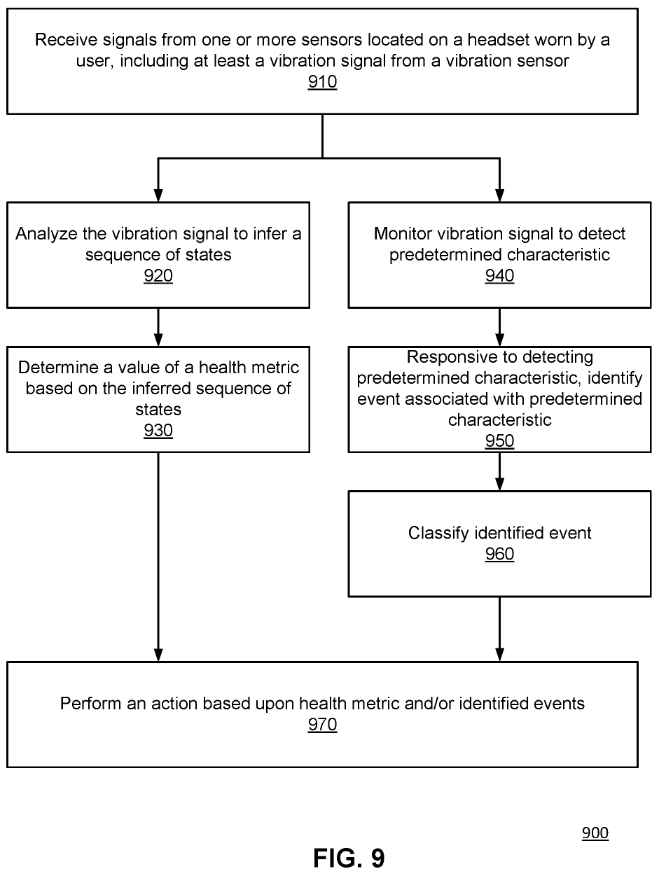

FIG. 9 is a flowchart of a method 900 for assessing a user condition with sensors mounted on a wearable device, according to some embodiments. In some embodiments, at least one or more of the steps in method 900 may be performed by a controller (e.g., the controller 804 illustrated in FIG. 8) comprising one or more processors. In some embodiments, the one or more processors include a processor executing instructions stored in a memory in either one of a smart glass, headset, or other wearable device on a user’s body part (e.g., head, arm, wrist, leg, ankle, finger, toe, knee, shoulder, chest, back, and the like). In some embodiments, the one or more processors include a processor executing instructions stored in a memory, wherein either the processor or the memory, or both, are part of a mobile device for the user, a remote server or a database, communicatively coupled with each other via a network. Moreover, the mobile device, the smart glass, and the wearable devices, etc. may be communicatively coupled with each other via a wireless communication system and protocol (e.g., radio, Wi-Fi, Bluetooth, near-field communication -NFC- and the like). In some embodiments, a method consistent with the present disclosure may include one or more steps from method 900 performed in any order, simultaneously, quasi-simultaneously, or overlapping in time.

The system receives 910 signals from one or more sensors located on a headset worn by a user, the signals including at least a vibration signal comprising vibration data collected by the vibration sensor. In some embodiments, the vibration signal comprises a waveform indicating vibration amplitude over a specific axis (e.g., z-axis oriented orthogonal to a surface of the sensor, where the surface of the sensor is configured to be substantially parallel to a surface on the user’s face, such as the side of the user’s nose when the headset is worn by the user) measured over time. In some embodiments, the vibration sensor is located within a nosepad of the headset, or within another portion of the headset that contacts the user’s body.

The system analyzes 920 the vibration signal to infer a sequence of states of the vibration signal. In some embodiments, the vibration signal is passed through a two-stage model, e.g., a first classification model and a second segmentation model. In some embodiments, the sequence of states corresponds to different states of respiration of the user, determined using the received vibration signal. In some embodiments, the system monitors the received vibration signal, and infers a sequence of states of the vibration signal as it is received.

The system determines 930 a value of a health metric based upon the inferred sequence of states of the monitored vibration signal. For example, where the sequence of states correspond to respiration states, the system may determine a respiratory rate of the user, based upon a timing of the determined sequence of states.

The system monitors 940 the vibration signal to detect a predetermined characteristic of the vibration, such as a predetermined trigger event. In some embodiments, the system may monitor the vibration signal for a plurality of different types of trigger events, each corresponding to a different type of event.

Responsive to detecting the predetermined characteristic, e.g., trigger event, the system identifies 950 an event associated with the predetermined characteristic. For example, the system may, responsive to detecting a trigger event, identify a portion of the vibration signal corresponding to a time period associated with the trigger event (e.g., a surrounding time period) to be associated with the identified event. In some embodiments, the time period may correspond to a predetermined period of time surrounding the detected characteristic (e.g., trigger event). In other embodiments, the time period may correspond to a time period before and after the detected characteristic that vibration signal continues to exhibit one or more predetermined characteristics (e.g., impulses having at least a threshold amplitude occurring at above a certain frequency). In some embodiments, the predetermined characteristics may be based upon the type of detected characteristic / trigger event.

The classifies 960 the identified event. For example, the system may, responsive to detecting the identified event, analyze the portion of the vibration signal associated with the identified event using the deep learning model to determine a type of event that occurred (e.g., respiratory event such as coughing or sneezing, eating/chewing events, drinking events, etc.). For example, in some embodiments, the event is classified based upon one or more of a frequency content, amplitude, and duration of the identified portion of the vibration signal associated with the event. In some embodiments, the event is classified using a decision tree model, based on the spectral and temporal features of the identified event.

In some embodiments, the system analyzes the received signals through a plurality of different pipelines, which may be executed in parallel. For example, as illustrated in FIG. 9, the system may analyze the vibration signal to infer a sequence of states and determine a value of a health metric based on the inferred sequence of states (steps 920 and 930) and monitor the vibration signal to detect a predetermined characteristic, whereupon an event associated with the predetermined characteristic is identified and classified (steps 940, 950, and 960) may be performed in parallel.

The system further performs 970 an action based upon the determined health metric and/or the identified event. For example, in some embodiments, the system may provide one or more recommendations to the user based on a respiratory health metric of the user, or generate one or more reports based upon a type of the one or more identified events, e.g., a report indicating the user’s current daily fluid intake following detection of a drinking event. In some embodiments, the system may combine or cross-reference the determined health metric and identified events, to generate a profile indicating an overall health characteristic of the user, e.g., combining the user’s monitored respiratory rate with information pertaining to detected coughing or sneezing events to generate a profile indicating an overall cardiovascular health of the user. The system may further perform additional actions (e.g.,. generate one or more notifications or reports to the user, select appropriate media to be played to the user, etc.) based upon the generated profile.

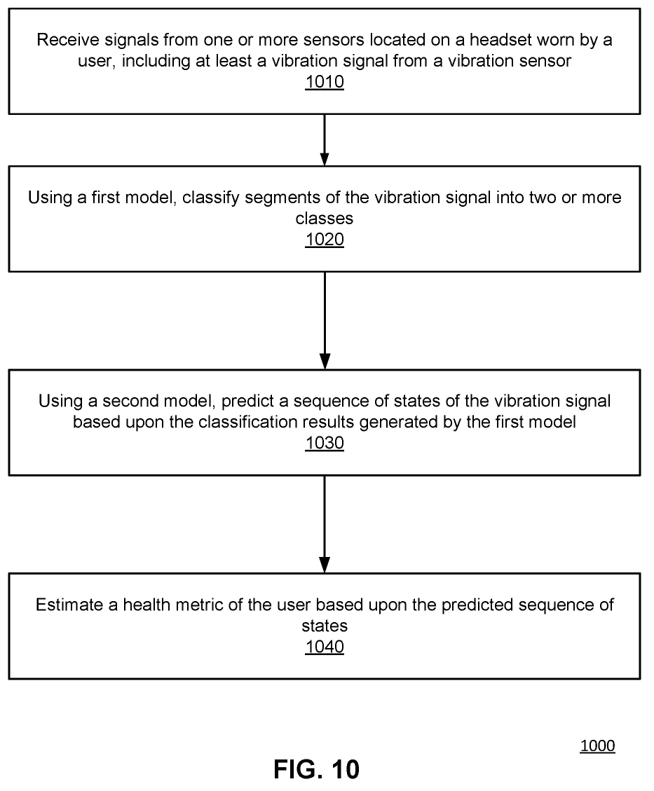

FIG. 10 is a flowchart of a method 1000 for determining a health metric for a user using a vibration sensor mounted a wearable device, in accordance with some embodiments. In some embodiments, at least one or more of the steps in method 1000 may be performed by a controller (e.g., the controller 804 illustrated in FIG. 8) comprising one or more processors. In some embodiments, the method 1000 may be performed as part of the first pipeline as described in relation to FIG. 9 (steps 920 and 930).

The system receives 1010 signals from one or more sensors located on a headset worn by a user, the signals including at least a vibration signal comprising vibration data collected by the vibration sensor. Using a first model, the system 1020 classifies segments of the received vibration signal into two or more classes. In some embodiments, the first model is a kNN model, an NB model, or a SVB. The two or more classes may correspond to different respiratory states of the user, such as a breathing state and a non-breathing state, or an inhale state, exhale state, and non-breathing state of the user.

The system uses a second model to predict 1030 a sequence of states of the vibration signal, based upon the classification results generated by the first model. In some embodiments, the second model comprises an HSMM that is configured to predict a most likely sequence of states based on the classification results and a priori knowledge of respiratory cycle characteristics (e.g., durations and transitions), by predicting times of highest probability of transitioning between different respiratory states.

The system estimates 1040 a health metric of the user based upon the predicted sequence of states. For example, in some embodiments, the system estimates a respiratory rate of the user based on the predicted sequence of respiratory states, e.g., based on a period of time between consecutive states of the same type.

### Example System Environment

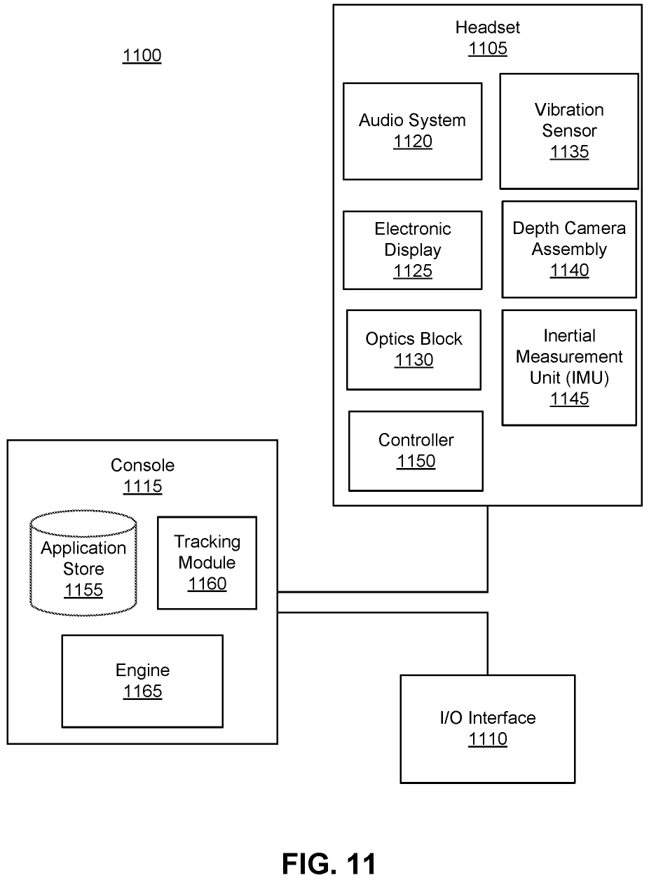

FIG. 11 is an example system environment of a headset including an audio system, in accordance with one or more embodiments. The system 1100 may operate in an artificial reality environment. The system 1100 shown in FIG. 11 includes a headset 1105 and an input/output (I/O) interface 1110 that is coupled to a console 1115. The headset 1105 may be an embodiment of the headset 100. While FIG. 11 shows an example system 1100 including one headset 1105 and one I/O interface 1110, in other embodiments any number of these components may be included in the system 1100. For example, there may be multiple headsets 1105 each having an associated I/O interface 1110 with each headset 1105 and I/O interface 1110 communicating with the console 1115. In alternative configurations, different and/or additional components may be included in the system 1100. Additionally, functionality described in conjunction with one or more of the components shown in FIG. 11 may be distributed among the components in a different manner than described in conjunction with FIG. 11 in some embodiments. For example, some or all of the functionality of the console 1115 is provided by the headset 1105.

In some embodiments, the headset 1105 may correct or enhance the vision of a user, protect the eye of a user, or provide images to a user. The headset 1105 may be eyeglasses which correct for defects in a user’s eyesight. The headset 1105 may be sunglasses which protect a user’s eye from the sun. The headset 1105 may be safety glasses which protect a user’s eye from impact. The headset 1105 may be a night vision device or infrared goggles to enhance a user’s vision at night. Alternatively, the headset 1105 may not include lenses and may be just a frame with an audio system 1120 that provides audio (e.g., music, radio, podcasts) to a user.

In some embodiments, the headset 1105 may be a head-mounted display that presents content to a user comprising augmented views of a physical, real-world environment with computer-generated elements (e.g., two dimensional (2D) or three dimensional (3D) images, 2D or 3D video, sound, etc.). In some embodiments, the presented content includes audio that is presented via an audio system 1120 that receives audio information from the headset 1105, the console 1115, or both, and presents audio data based on the audio information. In some embodiments, the headset 1105 presents virtual content to the user that is based in part on a real environment surrounding the user. For example, virtual content may be presented to a user of the eyewear device. The user physically may be in a room, and virtual walls and a virtual floor of the room are rendered as part of the virtual content. In the embodiment of FIG. 11, the headset 1105 includes an audio system 1120, an electronic display 1125, an optics block 1130, a vibration sensor 1135, a depth camera assembly (DCA) 1140, an inertial measurement (IMU) unit 1145, and a controller 1150. Some embodiments of the headset 1105 have different components than those described in conjunction with FIG. 11. Additionally, the functionality provided by various components described in conjunction with FIG. 11 may be distributed differently among the components of the headset 1105 in other embodiments or be captured in separate assemblies remote from the headset 1105.

The electronic display 1125 displays 2D or 3D images to the user in accordance with data received from the console 1115. In various embodiments, the electronic display 1125 comprises a single electronic display or multiple electronic displays (e.g., a display for each eye of a user). Examples of the electronic display 1125 include: a liquid crystal display (LCD), an organic light emitting diode (OLED) display, an active-matrix organic light-emitting diode display (AMOLED), some other display, or some combination thereof.

The optics block 1130 magnifies image light received from the electronic display 1125, corrects optical errors associated with the image light, and presents the corrected image light to a user of the headset 1105. The electronic display 1125 and the optics block 1130 may be an embodiment of the display element 110. In various embodiments, the optics block 1130 includes one or more optical elements. Example optical elements included in the optics block 1130 include: an aperture, a Fresnel lens, a convex lens, a concave lens, a filter, a reflecting surface, or any other suitable optical element that affects image light. Moreover, the optics block 1130 may include combinations of different optical elements. In some embodiments, one or more of the optical elements in the optics block 1130 may have one or more coatings, such as partially reflective or anti-reflective coatings.

Magnification and focusing of the image light by the optics block 1130 allows the electronic display 1125 to be physically smaller, weigh less, and consume less power than larger displays. Additionally, magnification may increase the field of view of the content presented by the electronic display 1125. For example, the field of view of the displayed content is such that the displayed content is presented using almost all (e.g., approximately 110 degrees diagonal), and in some cases, all of the user’s field of view. Additionally, in some embodiments, the amount of magnification may be adjusted by adding or removing optical elements.

In some embodiments, the optics block 1130 may be designed to correct one or more types of optical error. Examples of optical error include barrel or pincushion distortion, longitudinal chromatic aberrations, or transverse chromatic aberrations. Other types of optical errors may further include spherical aberrations, chromatic aberrations, or errors due to the lens field curvature, astigmatisms, or any other type of optical error. In some embodiments, content provided to the electronic display 1125 for display is pre-distorted, and the optics block 630 corrects the distortion when it receives image light from the electronic display 1125 generated based on the content.

The vibration sensor 1135 may comprise an accelerometer, a gyroscope (e.g., 1 axis, 2 axis, and/or three axis), a vibrometer, a contact microphone, some other sensor that can directly and/or indirectly be used to measure tissue vibration, or some combination thereof. In some embodiments, the vibration sensor 1135 may correspond to the vibration sensor 195 illustrated in FIG. 1, and be integrated into a portion of the headset 1105 that contacts the user’s body (e.g., within a nosepad that contacts the side of the user’s nose when the headset 1105 is worn by the user).

The DCA 1140 captures data describing depth information for a local area surrounding the headset 1105. In one embodiment, the DCA 1140 may include a structured light projector, an imaging device, and a controller. The imaging device may be an embodiment of the imaging device 120. The structured light projector may be an embodiment of the illuminator 125. The captured data may be images captured by the imaging device of structured light projected onto the local area by the structured light projector. In one embodiment, the DCA 1140 may include two or more cameras that are oriented to capture portions of the local area in stereo and a controller. The captured data may be images captured by the two or more cameras of the local area in stereo. The controller computes the depth information of the local area using the captured data. Based on the depth information, the controller determines absolute positional information of the headset 1105 within the local area. The DCA 1140 may be integrated with the headset 1105 or may be positioned within the local area external to the headset 1105.

The IMU 1145 is an electronic device that generates data indicating a position of the headset 1105 based on measurement signals received from one or more position sensors. A position sensor generates one or more measurement signals in response to motion of the headset 1105. Examples of position sensors 1135 include: one or more accelerometers, one or more gyroscopes, one or more magnetometers, another suitable type of sensor that detects motion, a type of sensor used for error correction of the IMU 1145, or some combination thereof. The position sensors may be located external to the IMU 1145, internal to the IMU 1145, or some combination thereof.

Based on the one or more measurement signals from one or more position sensors, the IMU 1145 generates data indicating an estimated current position of the headset 1105 relative to an initial position of the headset 1105. For example, the position sensors include multiple accelerometers to measure translational motion (forward/back, up/down, left/right) and multiple gyroscopes to measure rotational motion (e.g., pitch, yaw, and roll). In some embodiments, the IMU 1145 rapidly samples the measurement signals and calculates the estimated current position of the headset 1105 from the sampled data. For example, the IMU 1145 integrates the measurement signals received from the accelerometers over time to estimate a velocity vector and integrates the velocity vector over time to determine an estimated current position of a reference point on the headset 1105. Alternatively, the IMU 1145 provides the sampled measurement signals to the console 1115, which interprets the data to reduce error. The reference point is a point that may be used to describe the position of the headset 1105. The reference point may generally be defined as a point in space or a position related to the eyewear device’s 1105 orientation and position.

The IMU 1145 receives one or more parameters from the console 1115. As further discussed below, the one or more parameters are used to maintain tracking of the headset 1105. Based on a received parameter, the IMU 1145 may adjust one or more IMU parameters (e.g., sample rate). In some embodiments, data from the DCA 1140 causes the IMU 1145 to update an initial position of the reference point so it corresponds to a next position of the reference point. Updating the initial position of the reference point as the next calibrated position of the reference point helps reduce accumulated error associated with the current position estimated the IMU 1145. The accumulated error, also referred to as drift error, causes the estimated position of the reference point to “drift” away from the actual position of the reference point over time. In some embodiments of the headset 1105, the IMU 1145 may be a dedicated hardware component. In other embodiments, the IMU 1145 may be a software component implemented in one or more processors.

The controller 150 comprises one or more processors configured to receive vibration data generated by the vibration sensor 1135, and determine a value of health metric using the monitored tissue vibrations, wherein the health metric describes health and/or habits of the user, and may corresponding to physiological signals, health events, symptoms, emotional states, etc. As discussed above, the controller 1150 may input data from the vibration sensor 1135 into one or more models. In some embodiments, the one or more models may correspond to two or more different model pipelines, e.g., a first model pipeline that segments and classifies monitored vibration data to infer a series of states (e.g., respiratory states), and a second model pipeline for detecting events (e.g., responsive to detecting a trigger event) and classify detected events. In some embodiments, the controller 1150 may further cross-reference data from the vibration sensor 1135 with one or more other sensors on the headset 1105, such as an IMU, temperature sensor, blood oxygen sensor, camera, etc. In some embodiments, one or more operations of the controller 1150 may be implemented on the console 1115 or other electronic device (e.g., a mobile device).

The I/O interface 1110 is a device that allows a user to send action requests and receive responses from the console 1115. An action request is a request to perform a particular action. For example, an action request may be an instruction to start or end capture of image or video data, start or end the audio system 1120 from producing sounds, start or end a calibration process of the headset 1105, or an instruction to perform a particular action within an application. The I/O interface 1110 may include one or more input devices. Example input devices include: a keyboard, a mouse, a game controller, or any other suitable device for receiving action requests and communicating the action requests to the console 1115. An action request received by the I/O interface 1110 is communicated to the console 1115, which performs an action corresponding to the action request. In some embodiments, the I/O interface 1115 includes an IMU 1145, as further described above, that captures calibration data indicating an estimated position of the I/O interface 1110 relative to an initial position of the I/O interface 1110. In some embodiments, the I/O interface 1110 may provide haptic feedback to the user in accordance with instructions received from the console 1115. For example, haptic feedback is provided when an action request is received, or the console 1115 communicates instructions to the I/O interface 1110 causing the I/O interface 1110 to generate haptic feedback when the console 1115 performs an action.

The console 1115 provides content to the headset 1105 for processing in accordance with information received from one or more of: the headset 1105 and the I/O interface 1110. In the example shown in FIG. 11, the console 1115 includes an application store 1155, a tracking module 1160, and an engine 1165. Some embodiments of the console 1115 have different modules or components than those described in conjunction with FIG. 11. Similarly, the functions further described below may be distributed among components of the console 1115 in a different manner than described in conjunction with FIG. 11.

The application store 1155 stores one or more applications for execution by the console 1115. An application is a group of instructions, that when executed by a processor, generates content for presentation to the user. Content generated by an application may be in response to inputs received from the user via movement of the headset 1105 or the I/O interface 1110. Examples of applications include: gaming applications, conferencing applications, video playback applications, calibration processes, or other suitable applications.

The tracking module 1160 calibrates the system environment 1100 using one or more calibration parameters and may adjust one or more calibration parameters to reduce error in determination of the position of the headset 1105 or of the I/O interface 1110. Calibration performed by the tracking module 1160 also accounts for information received from the IMU 1145 in the headset 1105 and/or an IMU 1145 included in the I/O interface 1110. Additionally, if tracking of the headset 1105 is lost, the tracking module 1160 may re-calibrate some or all of the system environment 1100.

The tracking module 1160 tracks movements of the headset 1105 or of the I/O interface 1110 using information from the one or more sensor devices 1135, the IMU 1145, or some combination thereof. For example, the tracking module 1160 determines a position of a reference point of the headset 1105 in a mapping of a local area based on information from the headset 1105. The tracking module 1160 may also determine positions of the reference point of the headset 1105 or a reference point of the I/O interface 1110 using data indicating a position of the headset 1105 from the IMU 1145 or using data indicating a position of the I/O interface 1110 from an IMU 1145 included in the I/O interface 1110, respectively. Additionally, in some embodiments, the tracking module 1160 may use portions of data indicating a position or the headset 1105 from the IMU 1145 to predict a future location of the headset 1105. The tracking module 1160 provides the estimated or predicted future position of the headset 1105 or the I/O interface 1110 to the engine 1165.

The engine 1165 also executes applications within the system environment 1100 and receives position information, acceleration information, velocity information, predicted future positions, audio information, or some combination thereof of the headset 1105 from the tracking module 1160. Based on the received information, the engine 1165 determines content to provide to the headset 1105 for presentation to the user. For example, if the received information indicates that the user has looked to the left, the engine 1165 generates content for the headset 605 that mirrors the user’s movement in a virtual environment or in an environment augmenting the local area with additional content. Additionally, the engine 1165 performs an action within an application executing on the console 1115 in response to an action request received from the I/O interface 1110 and provides feedback to the user that the action was performed. The provided feedback may be visual or audible feedback via the headset 1105 or haptic feedback via the I/O interface 1110.

### Additional Configuration Information

The foregoing description of the embodiments has been presented for illustration; it is not intended to be exhaustive or to limit the patent rights to the precise forms disclosed. Persons skilled in the relevant art can appreciate that many modifications and variations are possible considering the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A headset, comprising:
a frame;
a vibration sensor coupled to the frame, the vibration sensor configured to monitor vibration of a tissue of a user wearing the headset; and
a controller configured to:
receive a signal corresponding to the monitored vibration from the vibration sensor;
analyze the received signal to infer a sequence of states of the received signal;
determine a value of a health metric based upon the inferred sequence of states, and
perform an action using the determined value of the health metric.

2. The headset of claim 1, wherein the vibration sensor is located within a nosepad of the frame.

3. The headset of claim 2, wherein the nosepad comprises an overmold configured to surround at least a portion of the vibration sensor.

4. The headset of claim 3, wherein the overmold contains a slot or cavity configured to accommodate the vibration sensor.

5. The headset of claim 3, wherein the vibration sensor is attached to a spring mounted within the overmold or to a flexible portion of the overmold.

6. The headset of claim 1, wherein the sequence of states corresponds to respiratory states of the user.

7. The headset of claim 6, wherein the controller is further configured to:
use a first model to classify segments of the received signal into two or more classes corresponding to different stages of respiration; and
use a second model to infer the sequence of states based upon the classification results generated by the first model.

8. The headset of claim 7, wherein the first model is a kNN model.

9. The headset of claim 7, wherein the second model is a hidden semi-Markov model (HSMM).

10. The headset of claim 6, where the controller is further configured to:
determine a respiratory rate of the user based upon the sequence of states; and
determine the health metric based upon at least in part upon the respiratory rate, wherein the health metric indicates a physical or emotional condition of the user.

11. The headset of claim 1, wherein the controller is further configured to:
monitor the received signal to detect a predetermined characteristic within the received signal;
responsive to detecting the predetermined characteristic:
identify a portion of the vibration signal corresponding to an event associated with the predetermined characteristic;
analyze the identified portion of the vibration signal to classify the identified event; and
perform an action based upon a type of the identified event.

12. The headset of claim 11, wherein the controller monitors the received signal to detect the predetermined characteristic in parallel with analyzing the received signal to infer a sequence of states of the received signal.

13. The headset of claim 11, wherein the event corresponds to an eating or drinking action by the user.

14. The headset of claim 13, wherein performing the action comprises notifying the user of a level of food or fluid consumption of the user, or of a food type consumed by the user.

15. The headset of claim 13, wherein the controller is further configured to monitor identified events over time to determine a habit of the user.

16. The headset of claim 11, wherein the controller is further configured to cross-reference the a value of the health metric determined based upon the inferred sequence of states with data associated with the identified event to determine a physical or emotional condition of the user.

17. The headset of claim 1, wherein the controller is further configured to determine, using the received signal, a tidal volume or respiratory flow rate of the user.

18. A computer-implemented method, comprising:
receiving, from a vibration sensor coupled to a frame of a headset, signal corresponding to a monitored vibration of a tissue of a user wearing the headset;
analyzing the received signal to infer a sequence of states of the received signal;
determining a value of a health metric based upon the inferred sequence of states, and
performing an action using the determined value of the health metric.

19. The computer-implemented method of claim 19, wherein the sequence of states corresponds to respiratory states of the user.

20. The computer-implemented method of claim 19, wherein analyzing the received signal to infer a sequence of states of the received signal comprises:
using a first model, classifying segments of the received signal into two or more classes corresponding to different stages of respiration; and
using a second model, inferring the sequence of states based upon the classification results generated by the first model.

21. The computer-implemented method of claim 19, wherein the first model is a kNN model, and the second model is a hidden semi-Markov model (HSMM).

22. The computer-implemented method of claim 18, further comprising:
monitoring the received signal to detect a predetermined characteristic within the received signal;
responsive to detecting the predetermined characteristic, analyzing the received signal to identify an event associated with the predetermined characteristic; and
performing an action based upon a type of the identified event.
